(19) United States
(12) Patent Application Publication
(10) Pub. No.: US 2016/0354509 A1
(43) Pub. Date: Dec. 8, 2016

(54) DEVICES FOR AND METHODS OF TREATMENT OF METABOLIC SYNDROMES

(71) Applicant: THE JOHNS HOPKINS UNIVERSITY, Baltimore, MD (US)

(72) Inventors: Michael Parlato, Baltimore, MD (US); Pratik Patel, Baltimore, MD (US); Kevin Colbert, Baltimore, MD (US); Ashish Nimgaonkar, Baltimore, MD (US); Pankaj Pasricha, Ellicott City, MD (US)

(21) Appl. No.: 15/117,715

(22) PCT Filed: Feb. 10, 2015
(86) PCT No.: PCT/US15/15260

§ 371 Date: Aug. 9, 2016

Related U.S. Application Data
(60) Provisional application No. 61/937,801, filed on Feb. 10, 2014.
(60) Provisional application No. 61/989,245, filed on May 6, 2014.

(51) Int. Cl. A61L 24/04

(52) U.S. Cl. CPC A61L 24/043

(57) ABSTRACT

Devices for and methods of treatment of metabolic syndromes are disclosed. Namely, the presently disclosed devices and methods are provided for affecting the function of the gastrointestinal endocrine system in key regions of the gut, thereby, producing therapeutic effects on obesity, diabetes and other metabolic syndromes. In some embodiments, the devices include multilayered bioadhesive patches delivered orally by dissolvable ingestible capsule, wherein various mechanisms are provided for anchoring the bioadhesive patches to the duodenal mucosa.

### CROSS-REFERENCE TO RELATED APPLICATIONS

The presently disclosed subject matter is related to U.S. Provisional Patent Application Nos. 61/937,801, filed Feb. 10, 2014, and 61/989,245, filed May 6, 2014, each of which is incorporated herein by reference in its entirety.

### TECHNICAL FIELD

The presently disclosed subject matter relates generally to medical devices and more particularly to devices for and methods of treatment of metabolic syndromes.

### BACKGROUND

Twenty-two percent of the 27 million patients with Type II diabetes mellitus (T2DM) in the United States are unable to control the disease despite being on intensive medical therapies. These patients are unable to keep their hemoglobin A1c (HbA1c), an indicator of average blood glucose levels over a 3-month period, below the American Diabetes Association (ADA) recommended 7% threshold and are at greater risk for heart disease, stroke and kidney disease. National Diabetes Statistics Report, 2014, Centers for Disease Control Prevention; Clark, 2008. Further, patients with diabetes sometimes hesitate to begin insulin therapy despite the clinical rationale for its necessity. One retrospective study of 80,000 cases showed that patients and providers hesitate 7 years on average before beginning insulin as a therapy. Khunti, K., et al., 2013.

Analysis of the typical diabetic patient's path from first line drugs to insulin and on to surgery and other last resort treatments reveals striking gaps, not limited to ineffective treatments and clinical inertia. Surgery and other solutions also have failed to achieve wide adoption. The addition of specialist clinicians in the care pathway has contributed to those failures. Accordingly, an effective treatment in the hands of the primary care physician would likely reach a much larger segment of the patient population than those which require a specialist, such as an endocrinologist, a gastroenterologist, or a surgeon.

### SUMMARY

In some aspects, the presently disclosed subject matter provides a method for applying a physical barrier to the gastrointestinal (GI) tract of a subject between the intestinal lining and the luminal contents, wherein the physical barrier has one or more of the following effects or characteristics: (a) is created in situ; (b) comprises one or more discrete and non-contiguous components; and (c) preserves significant nutrient absorption capacity within the intestines of the subject; and combinations thereof

In particular aspects, the physical barrier can be partial, discontinuous, discrete and spatially distributed, may have varying degrees of permeability, and may be present in varying amounts and regions of the GI tract. The physical barrier can include a bioadhesive component and may be delivered in the form of a plurality of patches, a syrup, a gel, a liquid, a powder, and combinations thereof and, in particular aspects, is non-absorbable and/or non-toxic.

In more particular aspects, the presently disclosed methods disrupt one or more signaling pathways in the GI tract by partially excluding a region thereof from contact with luminal contents. In other aspects, the one or more signaling pathways may be interrupted without significantly interfering with nutrient absorption.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 illustrates a perspective view of a bioadhesive patch that comprises a mucoadhesive, which is an example of the presently disclosed devices for partial exclusion of a portion of the GI tract from contact with luminal contents;

FIG. 2 illustrates a cross-sectional view of a portion of the GI tract that has a plurality of bioadhesive patches disposed therein;

FIG. 3A and FIG. 3B illustrate an example of using a capsule to deliver the bioadhesive patches shown in FIG. 1 to a portion of the GI tract;

FIG. 4A, FIG. 4B, FIG. 4C, and FIG. 4D illustrate a life cycle of another example of the bioadhesive patches deployed using a capsule, which is another example of the presently disclosed devices for partial exclusion of a portion of the GI tract from contact with luminal contents;

FIG. 5 illustrates an example of using an endoscope and/or catheter-like device to deliver the bioadhesive patches shown in FIG. 1 to a portion of the GI tract;

FIG. 6A and FIG. 6B illustrate an example of a folding bioadhesive patch, which is yet another example of the presently disclosed devices for partial exclusion of the GI tract from contact with luminal contents;

FIG. 7 illustrates a flow diagram of an example of a method of treating type 2 diabetes mellitus comprising the partial exclusion of a region of the GI tract from contact with luminal contents;

FIG. 8 illustrates a normal flow of luminal contents through a portion of gastrointestinal tract 800, e.g., a portion of the small intestine, through the stomach 812, duodenum 814, and jejunum 816;

FIG. 9 illustrates flow of luminal contents following RYGB surgery where the luminal contents, e.g., food particles, no longer pass through the duodenum 814, but instead pass through Roux limb 818, wherein the surgery interrupts or alters certain neurohormonal signaling pathways in the proximal intestine, leading to diabetic remission;

FIG. 10 illustrates preservation of nutrient absorption via a duodenal-jejunal bypass sleeve (DJBS) 1012 held in place by anchor 1010, e.g., a metal anchor, wherein, in this illustration nutrient absorption is blocked in duodenum 814 and jejunum 816 for 62 cm;

FIG. 11 illustrate preservation of nutrient absorption using the presently disclosed bioadhesive compositions 1110, in which in this representative example, a particular dosage can limit coverage to, for example, a 25-cm portion of the proximal intestines, thereby preserving nutrient absorption capacity. Key neurohormonal signaling pathways also are interrupted in this embodiment;

FIG. 12 is a flow diagram of a typical patient treatment pathway for a patent undergoing surgery for implantation of a duodenal-jejunal bypass sleeve (DJBS);

FIG. 13 is a flow diagram of a typical patient treatment pathway for a patent undergoing treatment with the presently disclosed bioadhesive compositions;

FIG. 14 illustrates a partial physical barrier 1400 comprising microspheres 1418, which create a partial lining along the villa 1414 of the mucosa 1412, which is atop the submucosa 1410, creating a partial barrier to luminal contents 1416;

FIG. 15 illustrates a partial physical barrier 1400 comprising patch 1510, which further comprises bioadhesive layer 1512 and backing layer 1510;

FIG. 16 illustrates a partial physical barrier 1400 comprising liquid bioadhesive 1610;

FIG. 17 illustrates a perspective view of a self-folding bioadhesive patch 1700 comprising non-adhesive layer 1725, swelling layer 1720, non-swelling layer 1715, mucoadhesive layer 1710, which can fold and attach to small intestine 1750;

FIG. 18, FIG. 19, FIG. 20, and FIG. 21 illustrate representative adhesion mechanisms of the presently disclosed bioadhesive patches including microsuckers 1810 (FIG. 18), barbs 1910 (FIG. 19), protrusions 2010 (FIG. 20), and micro-patterns 2110 (FIG. 21); and

FIG. 22 demonstrates that treatment with a mixture of polyvinylpyrrolidone and hyaluronic acid (PVP/HA) mucoadhesive showed significantly lowered postprandial blood glucose levels at the 30-, 45-, and 60-minute time points (p<0.05 in two repeated measure ANOVA) during a 2-hour oral glucose tolerance test. The treatment and control groups' blood glucose peaked at 30 minutes following oral gavage of glucose to 174% and 242% of baseline, respectively.

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

### I. Devices for and Methods of Treatment of Metabolic Syndromes

T2DM has traditionally been treated with insulin therapy and oral hypoglycemics, which compound over a patient's lifetime, cause weight gain, and lead to greater insulin resistance. Rubino, F., et al., 2009. More recently, approaches for treating T2DM derived from bariatric surgery have been pursued as alternatives to traditional diabetic treatments. While the scientific community continues to investigate the exact mechanism of action by which these approaches work, it is indisputable that duodenal bypass results in glucose homeostasis. As evidenced by randomized clinical trials, roux-en-y gastric bypass (RYGB) leads to the complete remission of T2DM in >80% of patients . Rubino, F., et al., 2009; Morton, J., 2013; Buchwald, H., et al., 2004; Buchwald, H., et al., 2009; Mingrone, G., et al., 2012; Brethauer, S.A., et al., 2013; Muñoz, R., et al., 2012; Boza, C., et al., 2011; Dixon, J.B., et al., 2011; Huang, C.-K., et al., 2011; Lee, W.-J., et al., 2011; de Sa, V. C. T., et al., 2011; Shah, S. S., et al., 2010; and Rubino, F. and M. Gagner, 2002.

While gastric bypass surgery is very effective, few patients meet the body mass index (BMI) requirements (greater than 35 kg/m²) and are willing to risk the complications of the major (and costly) operation. Fewer than 200,000 bariatric surgeries are performed each year for obese patients with T2DM. Nguyen, N. T., et al., 2011. Therefore, the remissive effects of the procedure are unavailable to 99% of diabetic patients. Other attempts have been made to provide alternative, less-invasive solutions with the introduction of the endoluminal sleeve and the mucosal resurfacing procedure, among others.

An example of the use of a bariatric sleeve to treat T2DM is a 62-cm flexible duodenal-jejunal bypass sleeve (DJBS) implanted in the intestines of a subject in need of treatment thereof for six to twelve months. The DJBS has been implanted in several thousands of patients in Europe, Chile, and Australia. GI Dynamics 2013 Annual Report. 2014. The device results in remission of T2DM in more than 60% of patients. de Moura, E. G., et al., 2011; de Moura, E., et al., 2012. The implant, however, can cause adverse effects including nausea, vomiting, and discomfort. Koehestanie, P., et al., 2014. The DJBS implantation also requires patients to take nutrient supplements following the procedure.

Other attempts have been made to take advantage of the effects of bariatric surgery in a less invasive manner. For example, international PCT patent application publication number WO2007115169 A3 to Bucevschi et al., describes the use of a swelling polymer to induce satiation. Bucevschi et al. also discloses use of a swelling polymer in treating diabetes, slowing the absorption of nutrients in the duodenum.

The above approaches share the goal of treating T2DM and/or obesity by limiting or inhibiting nutrient absorption. For instance, an ELS is described in U.S. patent application publication number 2012/0184967 A1 to Levine et al. discloses a method and apparatus for limiting absorption of food products in specific parts of the digestive system and mentions as an effect of bariatric surgery the shortening of “the effective-length of intestine available for nutrient absorption.” Further, as described in related U.S. Pat. No. 8,486,153, one aspect of this approach is that the “length is increased to further decrease absorption by bypassing a longer section of the jejunum,” further substantiating what appears to be the dependence of this approach on limiting nutrient absorption.

In contrast, the presently disclosed approach acts on a mechanism distinct from those approaches known in the art. Indeed, embodiments disclosed herein are designed to retain as much of the nutrient absorbing capacity of the duodenum as possible, while still resulting in a clinically significant effect on T2DM. This design consideration is made with the assumption that preserving the patient's ability to extract nutrients from sustenance is a desirable element of a treatment, and is based on the hypothesis that the primary mechanism in the efficacy of bariatric surgery is inhibition of neurohormonal signaling pathways as opposed to inhibited nutrient absorption.

As referred to herein, the phrase “preserving significant nutrient absorption” and variations thereof, is intended to mean retaining capacity for absorption such that the subject does not require nutritional supplements as a direct result of the treatment. Significant nutrient absorption may mean a level of nutrient absorption which is significantly higher when compared with those levels of nutrient absorption which occur in subjects as a result of procedures like the implantation of duodenal-jejunal bypass sleeve, for instance. Significant nutrient absorption should further be understood to include the absorption of nutrients such as carbohydrates, fats, proteins, vitamins or minerals. As disclosed herein below, “significant nutrient absorption” should further be understood to include such levels of nutrient absorption as might be demonstrable with a d-xylose blood absorption test in a normal subject.

Accordingly, the presently disclosed subject matter provides devices for and methods of treatment of metabolic disorders. As used herein, the term “metabolic disorder” includes glucose intolerance, pre-diabetes, type 1 and type 2 diabetes, obesity, dyslipidemia, hypertension and insulin resistance. Namely, the presently disclosed devices and methods are provided for affecting the function of the gastrointestinal endocrine system in particular regions of the duodenum, thereby, producing therapeutic effects on obesity, diabetes and other metabolic syndromes.

As used herein, the term “therapeutic effect” includes an effect or outcome that is desirable from the perspective of a physician, an effect which may be the goal of a procedure, such as, the implantation of the duodenal-jejunal bypass sleeve, a desirable effect that may be measured by a change in results on tests including, but not limited to, the fasting blood glucose, the oral glucose tolerance, hemoglobin Al c., and the like. The term “therapeutic effect” could further be construed to mean a desirable change in clinical parameters of a subject with a metabolic disorder.

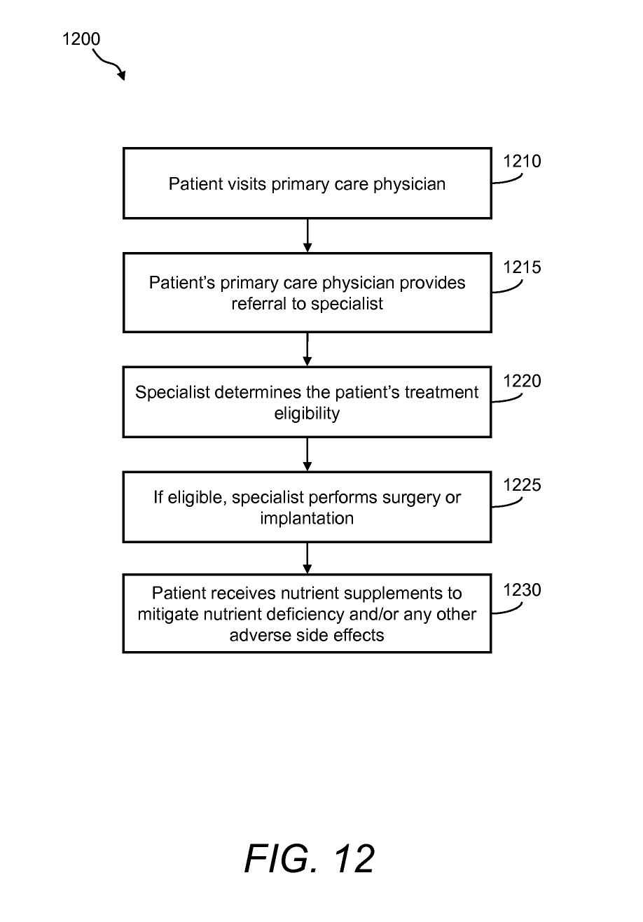

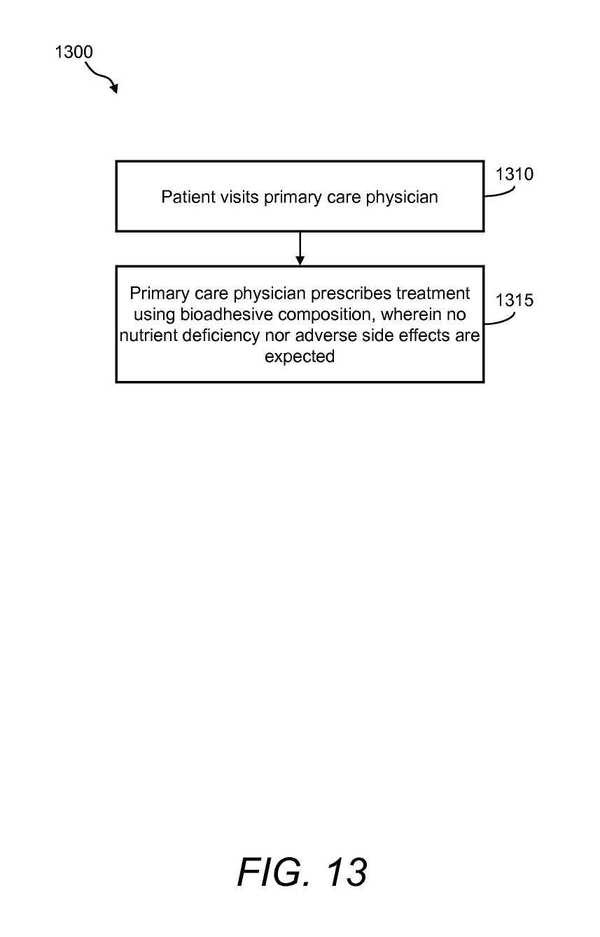

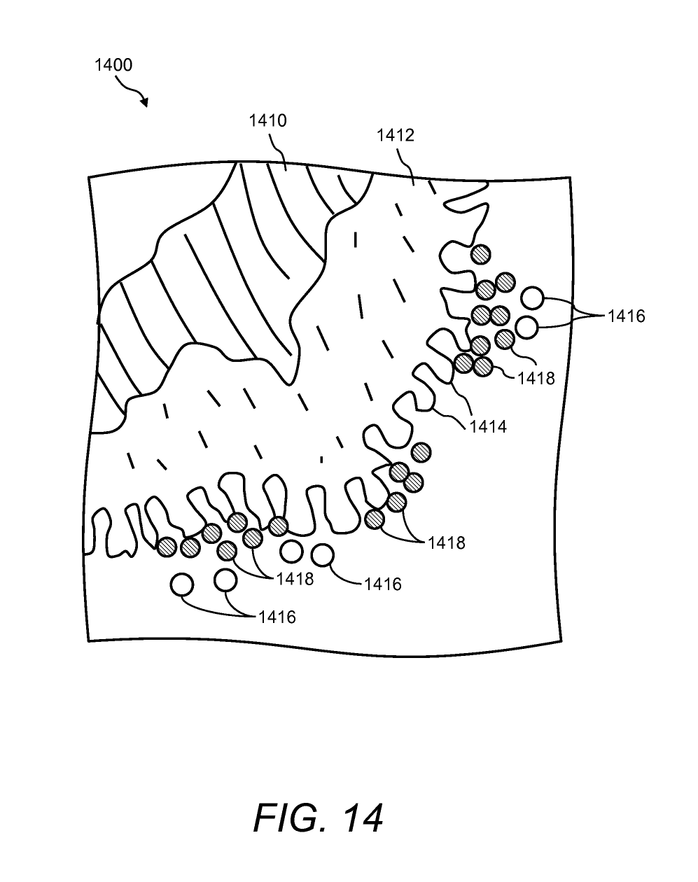

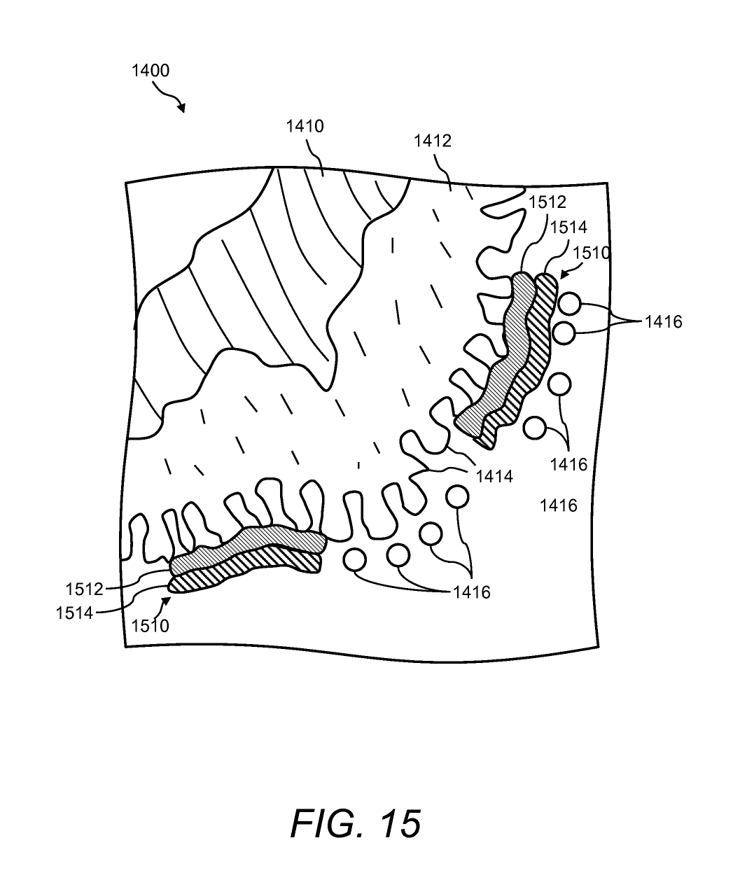

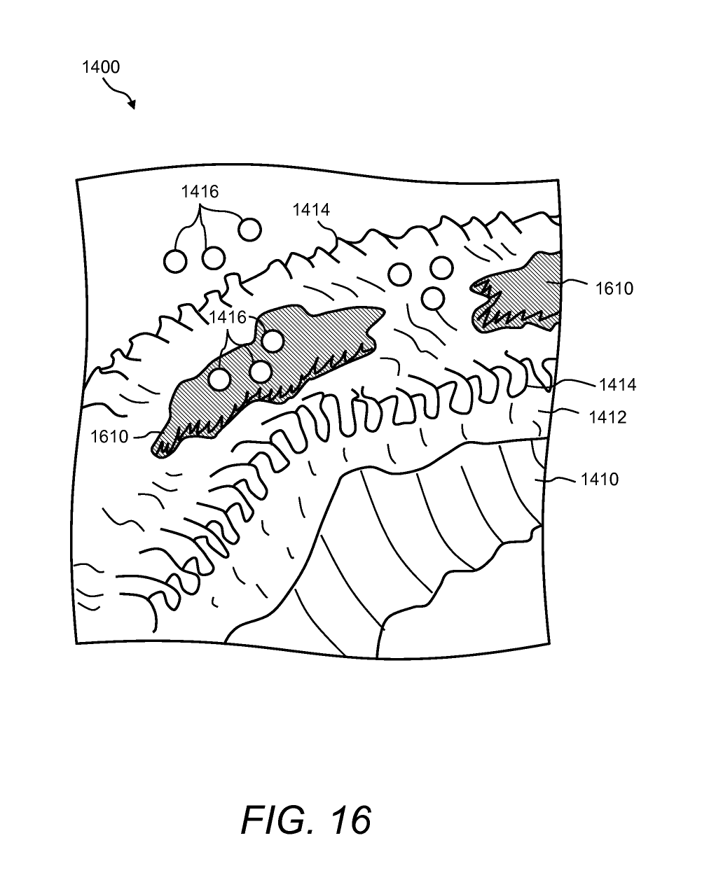

Compared to approaches known in the art, the presently disclosed subject matter requires less material; does not require circumferential coverage, i.e., coverage could be, in some embodiments, stochastic; does not interfere significantly with nutrient absorption; is generally noninvasive; and effectively removes additional stakeholders from the complicated T2DM care pathway (see FIG. 12).

Accordingly, in some embodiments, the presently disclosed subject matter provides a method for applying a physical barrier to the gastrointestinal (GI) tract of a subject between the intestinal lining and the luminal contents, wherein the physical barrier has one or more of the following effects or characteristics: (a) is created in situ; (b) comprises one or more discrete and non-contiguous components; and (c) preserves significant nutrient absorption capacity within the intestines of the subject; and combinations thereof.

As used herein, the term “intestinal lumen” refers to the cavity of the intestines. Further, as used herein, the terms luminal contents or, more particularly, intraluminal contents should be understood to include chyme, alimentary flow, nutrients, and food particles inside the intestinal lumen. As used herein, the term “proximal small intestines” should be understood to mean the portion of the intestines generally defined as the duodenum. Proximal small intestine should further be understood to include the first 0 to 50 cm of the small intestines following the stomach. That is, ranges such as the first 10 cm, the first 20 cm, the first 30 cm, and the first 40 cm of the human intestine should be included by proximal small intestines.

In particular embodiments, the physical barrier comprise a partial physical barrier. As used herein, the term “physical barrier” includes a structure that prevents the contact of one material with one or more other materials. For instance, a physical barrier may prevent the contact of the intraluminal contents (contents within the GI tract) with that of the lining and/or components within the wall of the GI tract. As used herein, the term “intestinal lining” refers to the lining of the wall of the GI tract which may comprise the mucosa and the mucus. Further, the term “partial” should be construed to mean less than 100%, discontinuous, discrete and spatially distributed, having varying degrees of permeability, or incomplete.

The physical barrier may be incomplete in preventing contact between one or more materials. Therefore, the physical barrier may further be partial, discontinuous, discrete and spatially distributed, may have varying degrees of permeability, and may be present in varying amounts and regions of the intestines. For instance, physical barrier may mean a semi-permeable liquid coating in contact with the mucus or mucins of the intestines. In other aspects of the present invention, physical barrier may refer to a plurality of discrete and spatially distributed amounts of material. In yet more particular embodiments, the method further comprises applying the physical barrier to limit an area of effective coverage on the lining of the GI tract to a section of the GI tract less than about 30 cm in length, wherein the section of the intestines begin at the pyloric sphincter and extends distally toward the jejunum. In particular embodiments, the physical barrier is applied to the GI tract starting in the duodenum with progressively increasing lengths from about 1 cm to about 30 cm, including 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 cm, including any integer and fraction thereof, to have a dose dependent effect.

In some embodiments, the physical barrier is created when a cationic compound combines in situ with the anionic mucins lining the wall of the intestines. As used herein, the term “creating in-situ” and variations thereof, implies enabling the formation of the final embodiment locally of the intended barrier from constituent elements at the desired site, rather than in a pre-assembled manner.

In representative embodiments, the physical barrier comprises a bioadhesive component. As used herein, the term “bioadhesive component” means any materials having adhesive properties which make them candidates for adhering to mucosa or mucins. Examples of bioadhesive materials include, but are not limited to, Carbopol®, cellulose, polycarbophil cysteine, poly (acrylic acid) derivatives, chemically modified poly (acrylic acids), polysaccharides, chitosan, chemically modified chitosan, cellulose, polycarbophil, cysteine, poly (acrylic acid), thiolated chitosan, poly(methacrylic acid) sodium salt, sodium alginate, sodium carboxymethylcellulose, sodium hyaluronate, hydroxyethylcellulose, hydroxypopylcellulose, polyvinylpirrolidone, polyethylene glycol, thiolated polymers, carboxymethylcellulose, dextran sulfate, hydroxyalkylcellulose, dermatan sulfate, water soluble vinyl polymer, bismuth, guar gum, xanthan gum, pectin and combinations thereof.

In particular embodiments, the bioadhesive component adheres to the mucosa of the GI tract for a retention period. As used herein, the term “retention period” includes time periods from ranging from half an hour to 7 days, including time period ranging from 1 hour to 3 hours, 1 hour to 5 hours, 1 hour to 24 hours, 1 to 3 days, and others.

In certain embodiments, the bioadhesive component is selected from the group consisting of a naturally occurring material and a synthetic material, or derivatives and combinations thereof. In some embodiments, the bioadhesive component comprises a naturally occurring material, or derivatives thereof, selected from the group consisting of cellulose, chitosan, chemically modified chitosan, cysteine, thiolated chitosan, sodium alginate, sodium carboxymethylcellulose, sodium hyaluronate, hydroxyethylcellulose, hydroxypropylcellulose, carboxymethylcellulose, dextran sulfate, hydroxyalkylcellulose, dermatan sulfate, guar gum, xanthan gum, pectin, and combinations thereof.

In other embodiments, the bioadhesive component comprises a synthetic material selected from the group consisting of polycarbophil cysteine, poly(acrylic acid) derivatives, chemically modified poly(acrylic acids), polycarbophil, poly (acrylic acid), poly(methacrylic acid) sodium salt, polyvinylpirrolidone, polyethylene glycol, thiolated polymers, water soluble vinyl polymer, and combinations thereof. In certain embodiments, the bioadhesive component comprises a layer having a thickness between about 0.1 microns and 1000 microns.

As provided herein below, in still yet other embodiments, the physical barrier, or a formulation or components thereof, further comprises a dissolvable substance, wherein the dissolvable substance dissolves at one or more specific sites, such as the pyloric antrum, pyloric canal, and pyloric sphincter, pylorus, of the gastrointestinal tract of the subject thereby applying the physical barrier thereto. In particular embodiments, the dissolvable substance dissolves in the stomach. In yet more particular embodiments, the dissolvable substance dissolves in the proximal small intestine. In representative embodiments, the dissolvable substance comprises a component selected from the group consisting of a methyl acrylate-methacrylic acid copolymer, a methyl methacrylate-methacrylic acid copolymer, a cellulose acetate succinate, a hydroxyl propyl methyl cellulose phthalate, a hydroxyl propyl methyl cellulose acetate succinate, a polyvinyl acetate phthalate, a cellulose acetate trimellitate, a sodium alginate, shellac, and combinations thereof.

In certain embodiments, the dissolvable substance dissolves at one or more specific sites, such as the stomach and/or proximal small intestine, in the gastrointestinal tract of the subject when contacted with an acidic substance or basic substance at a pH or range of pHs suitable for dissolving the substance.

In some embodiments, the dissolvable substance has a variable concentration within the physical barrier or formulation thereof. In particular embodiments, the dissolvable substance comprises a layer having a thickness between about 0.1 microns and 1000 microns. In yet more particular embodiments, the physical barrier or a formulation thereof comprises an expandable hydrogel adapted to apply pressure on the inner intestinal lumen of the subject thereby facilitating adhesion of the physical barrier to the intestinal lumen.

In some embodiments, the physical barrier further comprises a component selected from the group consisting of a semipermeable component, an impermeable component, an enteric component, and combinations thereof. In particular embodiments, the semipermeable or impermeable layer has a thickness between about 0.1 microns and 1000 microns.

In particular embodiments, the one or more impermeable or semipermeable bioadhesive materials comprise a plurality of discrete bioadhesive patches, which are described in more detail herein below, in which the plurality of discrete bioadhesive patches comprise one or more layers. The one or more layers can be selected from the group consisting of a backing layer, a mucoadhesive layer, an enteric layer, and combinations thereof. Generally, the term “patches” should be construed to mean discrete amounts of material which are spatially distributed from one another. For instance, patches may refer to solid circular particles with 2 or more layers which, when attached to the mucosa, are spatially distributed by a distance between 0.1 microns and 1000 microns. In other aspects, patches may refer to particles with 2 or more layers which are hydrated upon contacting the intestinal wall or intestinal contents.

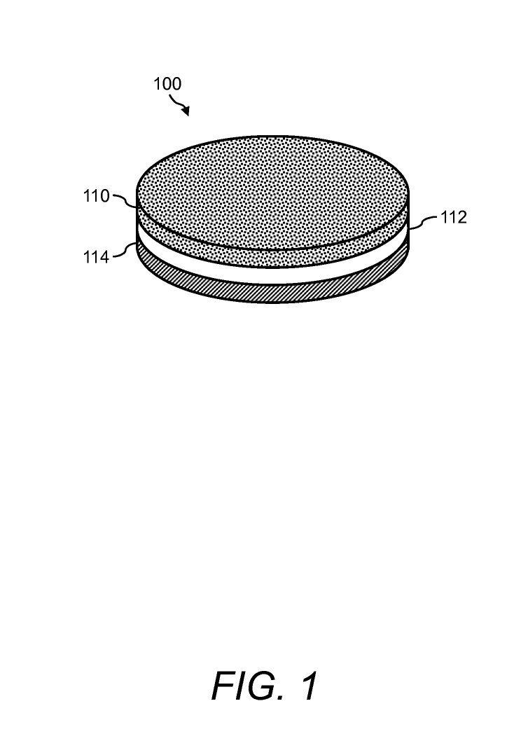

Referring now to FIG. 1 is a perspective view of a bioadhesive patch 100 that comprises a mucoadhesive, which is an example of the presently disclosed devices for partial exclusion of portions of the GI tract from contact with luminal contents. In this example, bioadhesive patch 100 comprises, in order, an occlusion layer 110, a mucoadhesive layer 112, and an enteral layer 114.

In some embodiments, occlusion layer 110 is an impermeable or semi-impermeable backing layer. Occlusion layer 110 can be, for example, from about 0.1 microns to about 1000 microns thick. Occlusion layer 110 can be formed, for example, of syrup, gel, liquid, powder, and any combinations thereof. In some embodiments, occlusion layer 110 can be ethylcellulose, cellulose acetate, and the like.

Mucoadhesive layer 112 is a bioadhesive material, such as high molecular weight homo- and co-polymers of acrylic acid, which can be crosslinked, for example, with a polyalkenyl, such as the Carbopol® family of polymers (The Lubrizol Company, Wickliffe, Ohio, USA), cellulose, polycarbophil cysteine, poly (acrylic acid) derivatives, chemically modified poly (acrylic acids), chitosan, chemically modified chitosan, and the like. Mucoadhesive layer 112 can be, for example, from about 0.1 microns to about 1000 microns thick.

Enteral layer 114 is a layer formed of a dissolvable substance, wherein the substance dissolves at a specific point or sites in the GI tract. Examples of dissolvable substances include, but are not limited to, poly(methyl) methacrylate-co-methacrylic acid) copolymer derivatives, hydroxypropyl methylcellulose phthalate, and the like. In some embodiments, the dissolvable enteral layer 114 dissolves when in contact with an additional acidic or basic substance at some specific pH. Enteral layer 114 can be, for example, from about 0.1 microns to about 1000 microns thick.

Bioadhesive patch 100 can have any shape or footprint. For example, bioadhesive patch 100 can be circular, ovular, square, rectangular, triangular, polygonal, and the like. In one example, bioadhesive patch 100 can be circular and has a diameter of from about 1 micron to about 5000 microns.

In operation, bioadhesive patch 100 is deployed into the subject's GI tract by various means. For example, bioadhesive patch 100 is taken into the subject's GI tract wherein a change in pH along the GI tract can cause enteral layer 114 of bioadhesive patch 100 to dissolve. In so doing, mucoadhesive layer 112 of bioadhesive patch 100 is exposed and adheres to the mucosa of the GI tract.

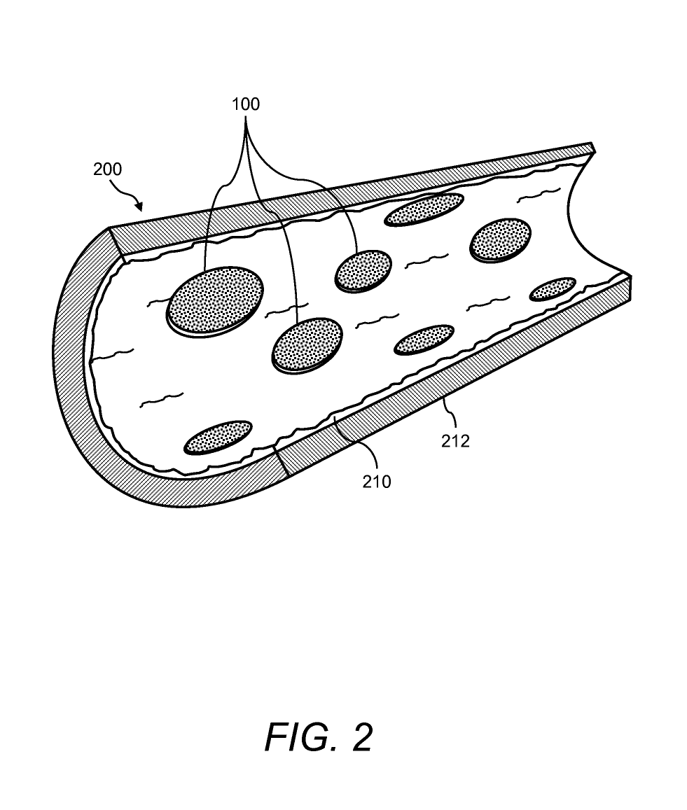

For example, FIG. 2 shows a cross-sectional view of a portion of the GI tract 200, e.g., a region of the duodenum, which has a plurality of bioadhesive patches 100 disposed on mucosa 210 of intestinal wall 212. Namely, bioadhesive patches 100 are anchored on mucosa 210 using mucoadhesive layer 112 and with occlusion layer 110 facing away from mucosa 210. Because occlusion layer 110 is an impermeable or semi-impermeable material, each bioadhesive patch 100 blocks an area of mucosa 210 from luminal contents. Bioadhesive patches 100 can adhere to mucosa 210 for, for example, a period of days or until they are eliminated. Accordingly, bioadhesive patches 100 can be used to treat type 2 diabetes mellitus because they create a partial exclusion of a region of the GI tract from contact with luminal contents.

In other embodiments, the physical barrier comprises a plurality of discrete microspheres. In particular embodiments, the discrete microspheres comprise one or more shells. In yet more particular embodiments, the discrete microspheres have a diameter between about 1 micron and about 1,000 microns.

In yet other embodiments, the one or more impermeable or semipermeable bioadhesive materials are selected from the group consisting of a syrup, a gel, a liquid, a powder, and combinations thereof.

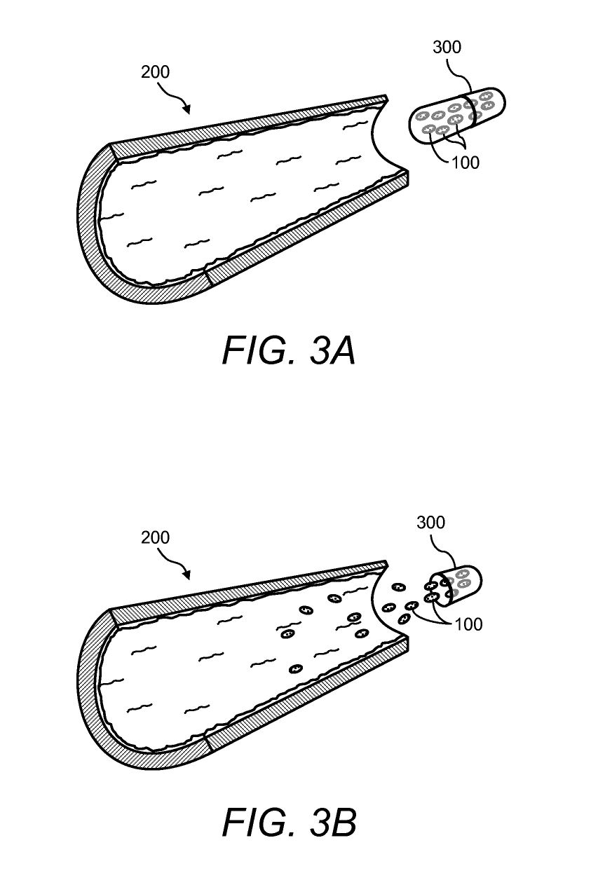

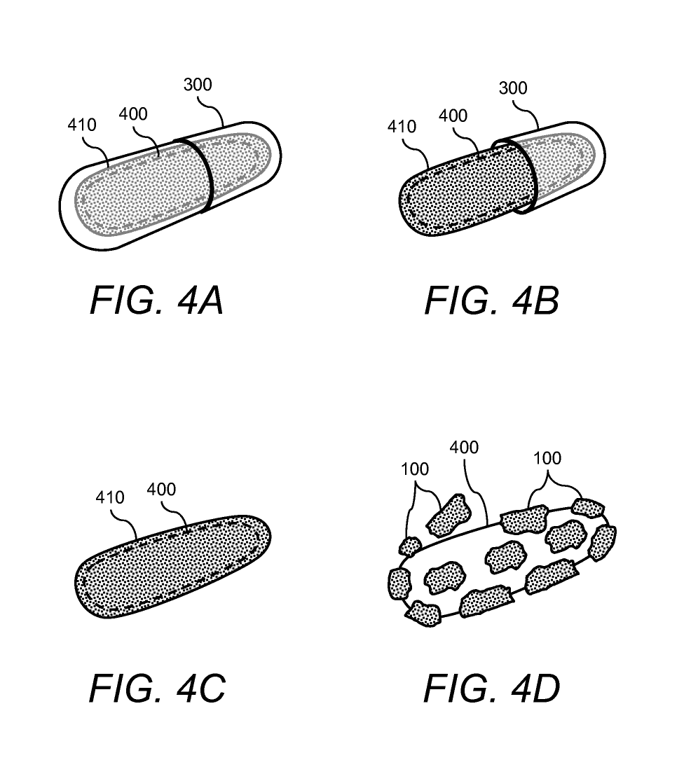

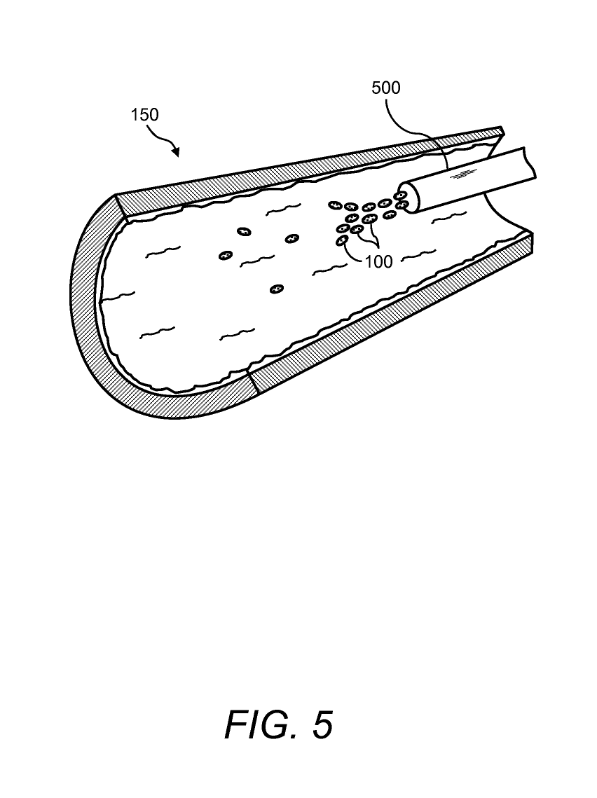

Bioadhesive patches 100, or syrups, gels, liquids, powders, and combinations thereof, can be deployed in various ways; examples of which are shown and described herein below with reference to FIG. 3A, FIG. 3B, FIG. 4A, FIG. 4B, FIG. 4C, FIG. 4D, and FIG. 5.

In other embodiments, the physical barrier is delivered to the gastrointestinal tract of the subject via an ingestible capsule. In some embodiments, the ingestible capsule has a hard or soft shell comprising one or more materials selected from the group consisting of animal protein, gelatin, a polysaccharide, starch, cellulose, a plasticizer, glycerin, sorbitol, a coloring agent, a preservative, a lubricant, and combinations thereof. Referring now to FIG. 3A and FIG. 3B is an example of using an ingestible capsule 300 to deploy bioadhesive patches 100 orally. For example, ingestible capsule 300 can be a dissolvable gelatin capsule that is holding a plurality of bioadhesive patches 100. Ingestible capsule 300 can be any ingestible capsule known in the art, and can include a hard or soft shell comprising one or more materials selected from the group consisting of animal protein, gelatin, a polysaccharide, starch, cellulose, a plasticizer, glycerin, sorbitol, a coloring agent, a preservative, a lubricant, and combinations thereof. In particular embodiments, the subject swallows ingestible capsule 300 (see FIG. 3A). Ingestible capsule 300 dissolves, for example, in the stomach or region of the small intestine, followed by the dispersion of bioadhesive patches 100 into GI tract 200 (see

FIG. 3B), e.g., a region of the duodenum. Again, a change in pH in the GI tract can cause enteral layer 114 of each bioadhesive patch 100 to dissolve and bioadhesive patches 100 adhere to the mucosa.

In some embodiments, the physical barrier is delivered to the gastrointestinal tract of the subject via an endoscope, a nasal or oral feeding tube, and combinations thereof. Referring now to FIG. 5 is an example of using an endoscope and/or catheter-like device 500 to deploy bioadhesive patch 100 into the subject's GI tract. For example, bioadhesive patch 100 is placed endoscopically with a catheter-like device, wherein the catheter has an opening at the distal end. The mucosa can be pulled by suction or other mechanism into the opening wherein bioadhesive patch 100 is attached thereto. Bioadhesive patch 100 being connected to the lining in such a way that bioadhesive patch 100 and tissue are released and bioadhesive patch 100 expands onto the released tissue.

In yet other embodiments, the physical barrier is delivered to the gastrointestinal tract of the subject via a formulation selected from the group consisting of an ingestible syrup, a liquid, a gel, an ointment, a powder, and a tablet.

In such embodiments, the one or more impermeable or semipermeable bioadhesive materials can be delivered to the gastrointestinal tract of the subject via an endoscope, a nasal or oral feeding tube, and combinations thereof. In further embodiments, the one or more impermeable or semipermeable bioadhesive materials are sprayed onto the inner intestinal lumen of the subject. In particular embodiments, the spraying is done endoscopically. In the liquid embodiments, the coating on the lining of the small intestines may be non-contiguous.

The one or more impermeable or semipermeable bioadhesive materials can comprise one or more components selected from the group consisting of ethylcellulose, cellulose acetate, natural and synthetic materials, and combinations thereof. In particular embodiments, at least one component of the one or more impermeable or semipermeable bioadhesive materials lining or its delivery mechanism comprises a dissolvable substance, wherein the dissolvable substance dissolves at one or more specific sites in the GI tract of a subject. In certain embodiments, the dissolvable substance comprises a poly(methyl)methacrylate-co-methacrylic acid copolymer derivative, hydroxypropyl methylcellulose phthalate, and combinations thereof. In yet more certain embodiments, the dissolvable substance dissolves at one or more specific sites in the GI tract of the subject when contacted with an acidic or basic substance at a pH or range of pHs suitable for dissolving the substance. In some embodiments, the dissolvable substance varies in thickness within the GI tract of the subject.

In particular embodiments, at least one component of the one or more impermeable or semipermeable bioadhesive materials attaches to tissue of the GI tract of the subject. In certain embodiments, at least one component of the one or more impermeable or semipermeable bioadhesive materials comprises a bioadhesive material selected from the group consisting of Carbopol®, cellulose, polycarbophil cysteine, poly (acrylic acid) derivatives, chemically modified poly (acrylic acids), chitosan, chemically modified chitosan, cellulose, polycarbophil, cysteine, poly(acrylic acid), thiolated chitosan, poly(methacrylic acid) sodium salt, sodium alginate, sodium carboxymethylcellulose, sodium hyaluronate, hydroxyethylcellulose, hydroxypopylcellulose, polyvinylpirrolidone, polyethylene glycol, thiolated polymers, and combinations thereof.

In some embodiments, at least one component of the one or more impermeable or semipermeable bioadhesive materials or its delivery mechanism comprises an expandable hydrogel adapted to apply pressure on the inner intestinal lumen of the subject thereby facilitating adhesion. Referring now to FIG. 4A, FIG. 4B, FIG. 4C, and FIG. 4D is a life cycle of another example of bioadhesive patches 100 deployed using a capsule and triggered by pH, which is another example of the presently disclosed devices for partial exclusion of a portion the GI tract from contact with luminal contents. In this example, enclosed in capsule 300 is an expandable core material 400 that is coated with a multilayer coating 410 (see FIG. 4A). In one example, expandable core material 400 is an expandable hydrogel. Multilayer coating 410 is formed substantially the same as bioadhesive patch 100, wherein multilayer coating 410 can comprise, in order, an occlusion layer, a mucoadhesive layer, and an enteral layer.

In a representative embodiment, the subject swallows capsule 300 and the gelatin capsule dissolves, for example, in the stomach (see FIG. 4B) and then expandable core material 400 with a multilayer coating 410 passes into the duodenum (see FIG. 4C). A change in pH between the stomach and the duodenum can cause expandable core material 400 to expand, which subsequently causes multilayer coating 410 to fracture into multiple segments and release from expandable core material 400, thereby forming a plurality of bioadhesive patches 100. Then, a change in pH between the stomach and the duodenum causes enteral layer 114 of occlusion patches 100 to dissolve and bioadhesive patches 100 adhere to the duodenal mucosa.

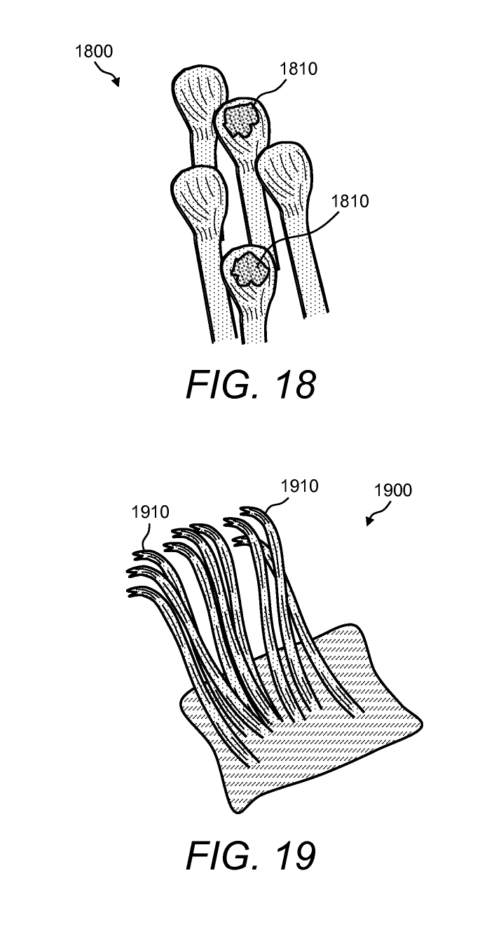

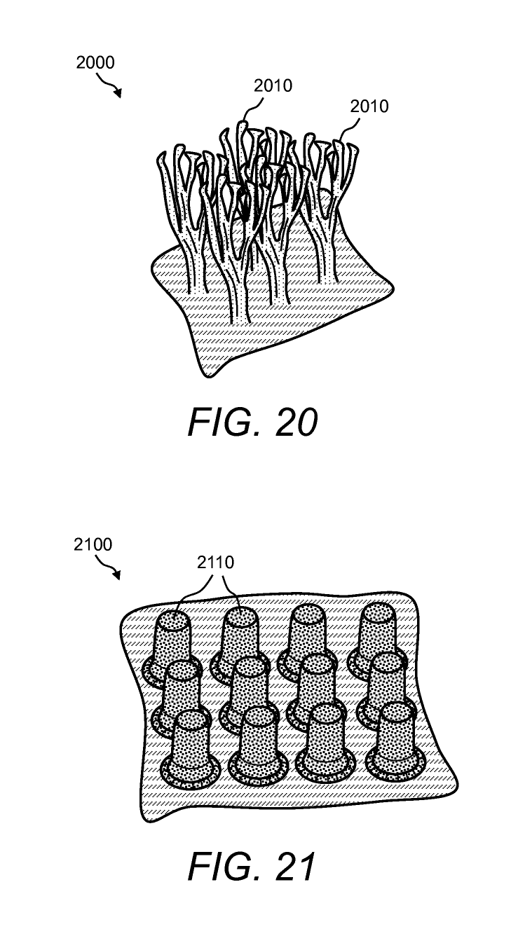

Bioadhesive patches 100 are not limited to attaching to the mucosa via mucoadhesive layer 112, which is a bioadhesive material. Other mechanisms can be used to attach bioadhesive patches 100 to the mucosa; examples of which are shown and described herein below with reference to FIGS. 18, 19, 20, and 21.

In some embodiments, at least one component of the one or more impermeable or semipermeable bioadhesive materials or its delivery mechanism reacts to a trigger mechanism thereby causing a folding of the lining comprising the one or more impermeable or semipermeable bioadhesive materials toward the inner intestinal lumen thereby facilitating adhesion. In particular embodiments, the trigger mechanism comprises a pH gradient.

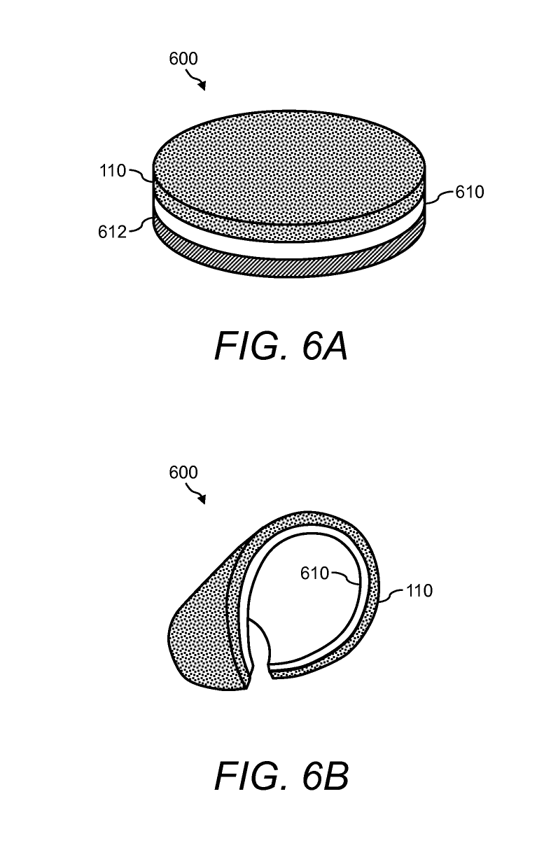

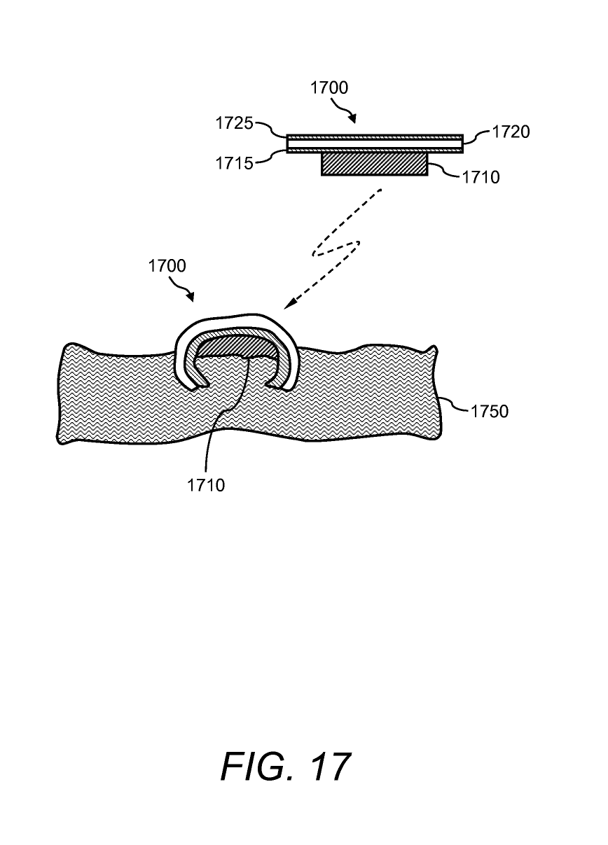

In other embodiments, the discrete patches are self-folding (see FIG. 6). Referring now to FIG. 17, in such embodiments, the discrete patches include a swelling layer. In particular embodiments, the swelling layer comprises a crosslinked poly(methyacrylic acid) hydrogel. In certain embodiments, the discrete self-folding patches include a non-swelling layer. In particular embodiments, the non-swelling layer comprises a poly(hydroxyethyl methacrylate) hydrogel.

In other embodiments, bioadhesive patch 600 can comprise a t-shaped anchor, which is yet another example of the presently disclosed devices for partial exclusion of a portion of the GI tract from contact with luminal contents.

In particular embodiments, the bioadhesive component includes an adhesion mechanism selected from the group consisting of one or more barbs, one or more micro-suckers, one or more protrusions, micro-patterning, and combinations thereof. Referring now to FIGS. 18-21 is a perspective view of a bioadhesive patches (1800, 1900, 2000, and 2100, respectively) that comprises anchor mechanisms including, one or more micro-suckers 1810 (FIG. 18), one or more barbs 1910 (FIG. 19), one or more protrusions 2010 (FIG. 20), and one or more micro-patterns 2110 (FIG. 21), and combinations thereof, which is still another example of the presently disclosed devices for partial exclusion of a portion the GI tract from contact with luminal contents.

In some embodiments, at least one component of the one or more impermeable or semipermeable bioadhesive materials or its delivery mechanism is passed by the natural digestive processes of the subject.

In other embodiments, at least one component of the one or more impermeable or semipermeable bioadhesive materials or its delivery mechanism is removable or reversible by the ingestion of a liquid solvent.

In particular embodiments, at least one component of the one or more impermeable or semipermeable bioadhesive materials or a formulation thereof is adapted to prevent or decrease adhesion of the lining to itself.

In yet other embodiments, the partial exclusion is achieved by submucosal injection of a material selected from the group consisting of a polymer, hydrogel, normal saline solution, glycerol, dextrose water, hyaluronic acid, polyvinylpyrrolidone fibrinogen mixture, hydroxypropyl methylcellulose, human albumin, polyvinyl alcohol, polyethylene glycol, hydroxyethyl starch, and combinations thereof.

In particular embodiments, the material remains in the GI tract of the subject for a period of time, thereby blocking contact of tissue in fluid communication with the mucosa with luminal contents.

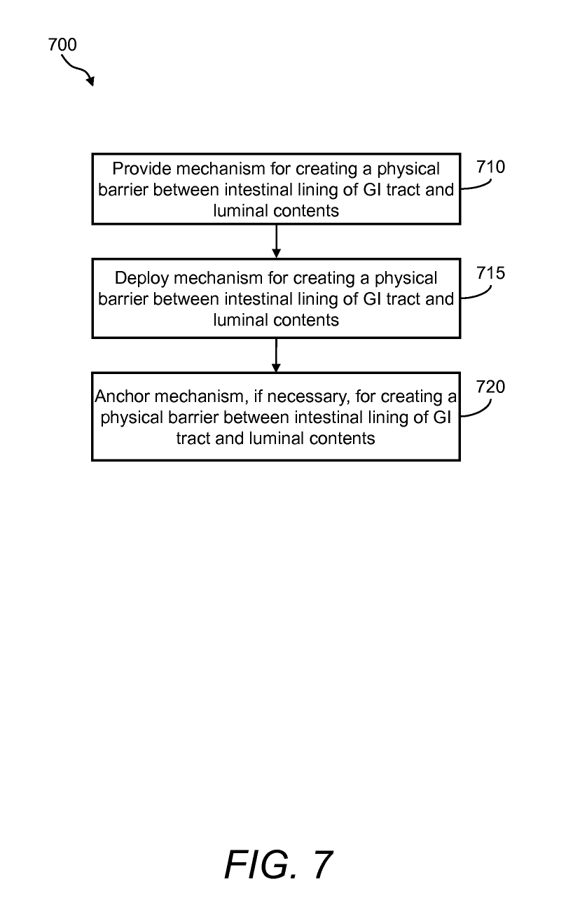

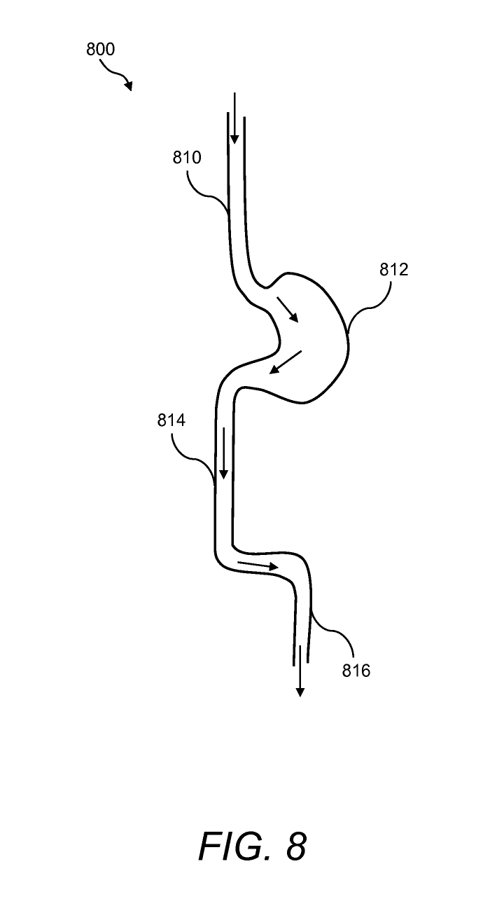

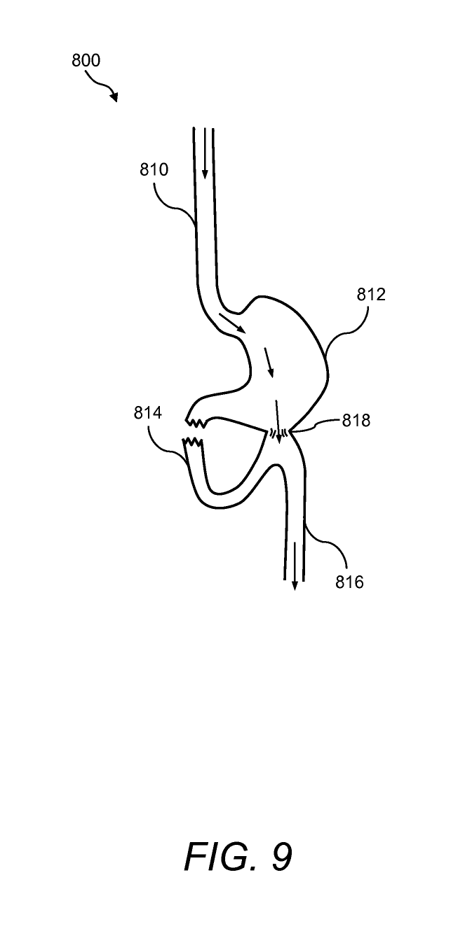

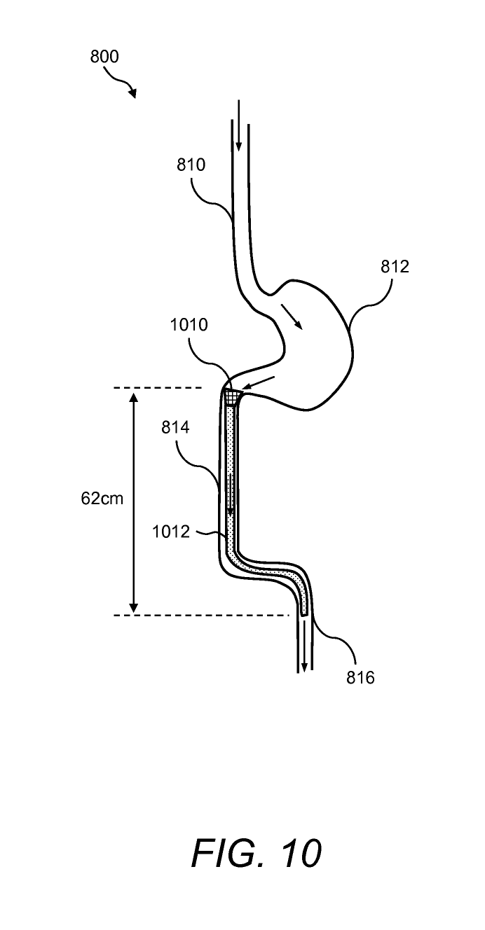

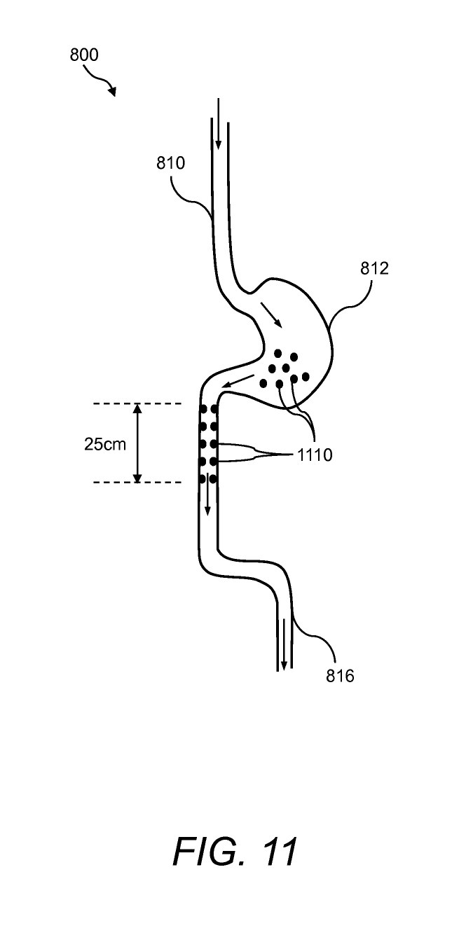

Referring now to FIG. 7 is a flow diagram of an example of a method 700 of treating type 2 diabetes mellitus comprising the partial exclusion of a portion of the GI tract from contact with luminal contents. Method 700 may include, but is not limited to, the following steps.

At a step 710, a mechanism for the partial exclusion of a portion the GI tract from contact with luminal contents is provided. For example, bioadhesive patches 100 as described with reference to FIG. 1 through FIG. 5 are provided.

At a step 715, the mechanism for the partial exclusion of the GI tract from contact with luminal contents is deployed. For example, bioadhesive patches 100 can be deployed into the GI tract orally via an orally ingestible dissolvable capsule, orally ingestible liquid carrier, or can be deployed endoscopically.

At a step 720, the mechanism for the partial exclusion of a portion of the GI tract from contact with luminal contents is anchored to the intestinal wall. For example, bioadhesive patches 100 are anchored to the intestinal wall via a bioadhesive layer.

In certain embodiments, the physical barrier or a formulation thereof is passed by natural digestive processes of the subject. In yet other embodiments, the physical barrier is removable or reversible by the ingestion of a liquid or solvent.

In some embodiments, the physical barrier comprises a liquid or a film.

In particular embodiments, the physical barrier and formulations thereof are non-toxic.

In particular embodiments, no component of the physical barrier is absorbed from the gastrointestinal tract during the formation of the physical barrier or retention period thereafter.

In some embodiments, the physical barrier, when compared to a sham control, does not result in a significant difference in blood d-xylose concentration as measured in a d-xylose absorption test.

In particular embodiments, the physical barrier comprises about 1% to about 5% by weight glycerine, including about 1.0, 2.0, 3.0, 4.0, and 5.0%, including any integers and fractions thereof, about 0.1% to about 5% by weight polyacrylic acid polymer, including about 0.1, 0.5, 1.0, 2.0, 3.0, 4.0, and 5.0%, including any integers and fractions thereof, about 0.1% to about 5% by weight potassium hydroxide, including about 0.1, 0.5, 1.0, 2.0, 3.0, 4.0, and 5.0%, including any integers and fractions thereof, and about 1.5% by weight benzyl alcohol. In yet more particular embodiments, the physical barrier comprises between about 0.1 wt/wt % to about 2.0 wt/wt %, including 0.1, 0.5, 1.0, 1.5, and 2.0, including any integers and fractions thereof, of one or more mucoadhesives, wherein the one or more mucoadhesive are selected from the group consisting of polyvinylpyrrolidone, carboxymethylcellulose, dextran sulfate, hydroxyalkylcellulose, dermatan sulfate, a water-soluble vinyl polymer, chitosan, guar gum, xanthan gum, tragacanth gum, pectin, and polyacrylic acid.

In other embodiments, the physical barrier comprises between about 0.1 wt/wt % to about 3.0 wt/wt %, including 0.1, 0.5, 1.0, 1.5, 2.0, 2.5, and 3.0%, including any integers and fractions thereof, of one or more mucoadhesives in an aqueous solution, wherein the one or more mucoadhesives comprise one or more linear or cross-linked polymers selected from the group consisting of polyacrylic acid, carboxymethylcellulose, hydroxyalkylcellulose, dextran sulfate, chitosan, and a water-soluble vinyl polymer.

In yet other embodiments, the physical barrier comprises about 0.35 wt/wt % of one or more mucoadhesives, and about 5 wt/wt % of a viscosity inducing agent in an aqueous solution. In still yet other embodiments, the physical barrier comprises about 0.35 wt/wt % of a polyacrylic acid polymer, 1.5 wt/wt % of benzyl alcohol, 0.4 wt/wt % of sodium saccharin, and 0.05% wt/wt % of polysorbate 60 in an aqueous solution. As used herein, a viscosity inducing agent is a substance that makes a formulation more viscous.

In still yet other embodiments, the physical barrier comprises: from about 0.001% to about 7%, including 0.001, 0.01, 0.1, 0.5, 1.0, 1.5, 2.0, 2.5, 3.0, 3.5, 4.0, 4.5, 5.0, 5.5, 6.0, 6.5, and 7.0%, including any integers and fractions thereof, by weight of hyaluronic acid, or a pharmaceutically acceptable salt thereof, wherein the hyaluronic acid has a molecular weight between about 1 million Daltons and 3 million Daltons; from about 0.01% to about 20% by weight, including 0.01, 0.1, 0.5, 1.0, 1.5, 2.0, 3.0, 4.0, 5.0, 10, and 20%, including any integers and fractions thereof, of a K60 to K100 polyvinylpyrrolidone; or a pharmaceutically acceptable salt thereof.

In other embodiments, the physical barrier comprises: from about 0.04% to about 5%, including 0.04, 0.1, 0.5, 1.0, 2.0, 3.0, 4.0, and 5%, including any integers and fractions thereof, by weight of hyaluronic acid, or a pharmaceutically acceptable salt thereof, wherein the hyaluronic acid has a molecular weight between about 1.6 to about 2.2 million Daltons; from about 0.08% to about 15% by weight, including 0.08, 0.1, 0.5, 1.0, 1.5, 2.0, 3.0, 4.0, 5.0, 10, and 15%, including any integers and fractions thereof, of a K60 to K100 polyvinylpyrrolidone; or a pharmaceutically acceptable salt thereof.

In some embodiments, no component of the physical barrier is absorbed from the gastrointestinal tract during the formation of the physical barrier or retention period thereafter. In particular embodiments, each of the components of the physical barrier are delivered in non-toxic doses.

In some embodiments, the presently disclosed subject matter provides a method for treating type 2 diabetes mellitus and/or obesity in a subject in need of treatment thereof, the method comprising disrupting one or more signaling pathways in the GI tract, e.g., in the proximal small intestine or duodenum, by partially excluding a region thereof from contact with luminal contents. As used herein, the term “neurohormonal signaling pathways” means communication from one region of the body to another via either nerves or secreted substances such as hormones. In some embodiments, the partial exclusion is achieved by lining a region of the duodenum with one or more impermeable or semipermeable bioadhesive materials as disclosed herein. In particular embodiments, the partial exclusion is stochastically distributed within the duodenum or proximal small intestine. Importantly, the presently disclosed methods disrupt one or more signaling pathways in the GI tract by partially excluding a region thereof from contact with luminal contents without significantly interfering with nutrient absorption.

In some embodiments, the metabolic disorder is selected from the group consisting of obesity, pre-diabetes, insulin resistance, type 1 diabetes mellitus, type 2 diabetes mellitus, glucose impairment, hypertension, dyslipidemia, and hyperlipidemia.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

In particular embodiments of the presently disclosed methods, the subject is a mammalian subject.

In yet other embodiments, the presently disclosed subject matter provides a method for marketing a treatment for a metabolic disorder comprising packaging the treatment along with labeling that identifies the treatment as being useful to: create a physical barrier in a subject between the intraluminal contents and the intestinal lining; preserves significant nutrient absorption capacity in the proximal intestines of the subject;

and/or inhibit one or more neurohormonal pathways in the proximal intestines of the subject.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following. Examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner to make compounds of the disclosure by other methods.

### Example 1

### Efficacy of Mucoadhesive Compounds as a Treatment for Type 2 Diabetes

### Methods

Testing was conducted to investigate the efficacy of a mucoadhesive lining in the treatment of type 2 diabetes. Infusion catheters were surgically implanted in Sprague

Dawley adult male rats (250 g-300 g). The catheters ran from the intestinal lumen one centimeter distal to the pylorus and exited out of the animal's upper back. After implantation of the infusion catheter, animals were allowed to recover for at least one week and housed individually at 19° C.-22° C. and 40%-60% humidity with a 12-hour light-dark cycle. Prior to experiment, all rats were fasted for 18 hours and allowed water.

Animals were split into a control group, which was gavaged with 0.9% saline and a treatment group, which was gavaged with 0.9 g/kg rat with a 0.1% by weight polyvinylpyrrolidone (PVP) and 9% by weight hyaluronic acids (HA) based mucoadhesive compound. The infusions were administered through the duodenal cannula in four equal doses separated by 30 minute intervals. Half an hour after the fourth gavage of saline or PVP/HA, baseline blood glucose levels were taken. Oral gavage of 0.1 g/mL glucose solution (1 g/kg rat) was given immediately following the baseline blood glucose reading. Glucose tolerance test samples were taken from each rat at 0, 15, 30, 45, 60, 75, 90, 105 and 120 minutes after the glucose administration.

### Results

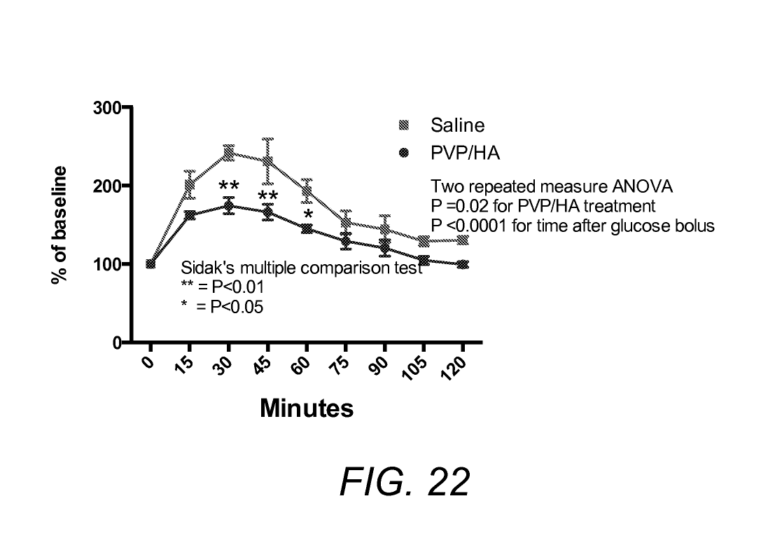

Treatment with PVP/HA mucoadhesive showed significantly lowered blood glucose levels at the 30, 45 and 60 minute time points (p<0.05 in two repeated measures ANOVA). The treatment and control groups' blood glucose peaked at 30 minutes following oral gavage of glucose to 174% and 242% of baseline, respectively (FIG. 22).

### Example 2

### Prophetic

### Preservation of Nutrient Absorption Capacity with Mucoadhesive Compounds

### Methods

Testing is conducted to investigate the advantage of select mucoadhesive compounds in their ability to demonstrate efficacy as a treatment for Type 2 Diabetes whilst allowing sufficient nutrient absorption when compared to other surgical and device-based treatment alternatives. Sprague Dawley adult male rats (250 g-300 g) are housed individually at 19° C.-22° C. and 40-60% humidity with a 12-hour light-dark cycle. Prior to experimentation, all rats are fasted for 18 hours and allowed water. Animals are split into two groups, a control group, which is gavaged with 0.9% saline, and a treatment group, which is gavaged with 0.9 g/kg of the PVP/HA-based mucoadhesive compound, each through the infusion cannula. Blood is taken at 0, 15, 30 and 45 minutes following the administration of the mucoadhesive or saline solution. The blood is stored in 15-mL vials and placed on ice for the duration of the collection.

Phloroglucinol (1,3,5-trihydroxybenzene) and D-xylose are obtained from Sigma Chemical Co (St. Louis, Mo. 63178). A color reagent consisting of 0.5 g of phloroglucinol, 100 mL of glacial acetic acid, and 10 mL of concentrated hydrochloric acid is prepared at most 48 hours prior to the experiment and protected from light. D-xylose is dissolved in saturated benzoic acid to make 0.325, 0.65, 1.3, 2.6, and 5.2 mmol/L concentrations of d-xylose standard solutions. 50 μL of plasma and xylose solutions is placed in disposable test tubes containing 5 mL of phloroglucinol color reagent. All tubes are heated for exactly 4 minutes at 100° C., then cooled to room temperature in water. A spectrophotometer is adjusted to zero absorbance with a reagent blank containing water (50 μL) and phloroglucinol reagent (5.0 mL) before reading the standard solutions, and with a serum blank (50 μL of xylose-free serum plus 5.0 mL of phloroglucinol reagent) before reading the absorbances of the tubes containing serum or plasma. Statistical calculations are performed by calculating the paired Student's t-test and correlation coefficients by standard methods. See Eberts, Thomas J., et al. “A simplified, colorimetric micromethod for xylose in serum or urine, with phloroglucinol.” Clinical Chemistry 25.8 (1979): 1440-1443.

### Example 3

### Prophetic

### Investigating the Effective Coverage Area of Mucoadhesive Compounds

### Methods

Testing is conducted to investigate the distribution of the mucoadhesive compound in the small intestine. Infusion catheters are surgically implanted in Sprague Dawley adult male rats (250 g-300 g). The catheters extend from the intestinal lumen one centimeter distal to the pylorus and exit out of the animal's upper back. After implantation of the infusion catheter, animals are allowed to recover for at least one week and housed individually at 19° C.-22° C. and 40-60% humidity with a 12-hour light-dark cycle. Prior to experiment, all rats are fasted for 18 hours and allowed water. Animals were then split into three groups, a control group is gavaged with 0.9% saline and a treatment group is gavaged with 0.9 g/kg rat PVP-HA based mucoadhesive compound with fluorescent tag, each through the infusion cannula in a single dose.

Animals are sacrificed 50 minutes after administration of the infusion. The gastrointestinal tract of each animal is put on ice immediately following dissection. The intestinal tract is partitioned in 5-cm increments along its length from the pylorus to the ileocecal valve. The tissue sections are filleted and laid flat with the lumen upwards. The mucosa is scraped and contents placed into disposable test tubes. A colorometric analysis at 488 nm is performed to compare the sections within an animal and between experimental groups.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
1. National Diabetes Statistics Report. 2014, Centers for Disease Control Prevention: www.cdc.gov.
2. Diabetes report card 2012. 2012, Centers for Disease Control Prevention.
3. Rubino, F., et al., Diabetes Surgery: a new approach to an old disease. Diabetes Care, 2009. 32(suppl 2): p. S368-S372.
4. Morton, J., Comment on: Outcomes of bariatric surgery in patients with BMI less than 35 kg/m2. Surgery for Obesity and Related Diseases, 2013. 9(1): p. 150.
5. Buchwald, H., et al., Bariatric surgery: a systematic review and meta-analysis. JAMA, 2004. 292(14): p. 1724-1737.
6. Buchwald, H., et al., Weight and type 2 diabetes after bariatric surgery: systematic review and meta-analysis. The American Journal of Medicine, 2009. 122(3): p. 248-256. e5.
7. Mingrone, G., et al., Bariatric surgery versus conventional medical therapy for type 2 diabetes. New England Journal of Medicine, 2012. 366(17): p. 1577-1585.
8. Brethauer, S. A., et al., Can diabetes be surgically cured? Long-term metabolic effects of bariatric surgery in obese patients with type 2 diabetes mellitus. Annals of Surgery, 2013. 258(4): p. 628-637.
9. Munoz, R., et al., Isolated duodenal exclusion increases energy expenditure and improves glucose homeostasis in diet-induced obese rats. American Journal of Physiology-Regulatory, Integrative and Comparative Physiology, 2012. 303(10): p. R985-R993.
10. Boza, C., et al., Safety and efficacy of Roux-en-Y gastric bypass to treat type 2 diabetes mellitus in non-severely obese patients. Obesity Surgery, 2011. 21(9): p. 1330-1336.
11. Dixon, J. B., et al., Bariatric surgery: an IDF statement for obese Type 2 diabetes. Diabetic Medicine, 2011. 28(6): p. 628-642.
12. Huang, C.-K., et al., Laparoscopic Roux-en-Y gastric bypass for the treatment of type II diabetes mellitus in Chinese patients with body mass index of 25-35. Obesity Surgery, 2011. 21(9): p. 1344-1349.
13. Lee, W.-J., et al., Diabetes remission and insulin secretion after gastric bypass in patients with body mass index<35 kg/m2. Obesity Surgery, 2011. 21(7): p. 889-895.
14. de Sa, V. C. T., et al., Gastric bypass in the treatment of type 2 diabetes in patients with a BMI of 30 to 35 kg/m2. Obesity Surgery, 2011. 21(3): p. 283-287.
15. Shah, S. S., et al., Diabetes remission and reduced cardiovascular risk after gastric bypass in Asian Indians with body mass index<35 kg/m<sup>2</sup>. Surgery for Obesity and Related Diseases, 2010. 6(4): p. 332-338.
16. Rubino, F. and M. Gagner, Potential of surgery for curing type 2 diabetes mellitus. Annals of Surgery, 2002. 236(5): p. 554.
17. Nguyen, N. T., et al., Trends in use of bariatric surgery, 2003-2008. Journal of the American College of Surgeons, 2011. 213(2): p. 261-266.
18. Grabovac, V., D. Guggi, and A. Bernkop-Schnürch, Comparison of the mucoadhesive properties of various polymers. Advanced Drug Delivery Reviews, 2005. 57(11): p. 1713-1723.
19. Chary, R., G. Vani, and Y. M. Rao, In vitro and in vivo adhesion testing of mucoadhesive drug delivery systems. Drug development and industrial pharmacy, 1999. 25(5): p. 685-690.
20. Saydah, S. H., J. Fradkin, and C. C. Cowie, Poor control of risk factors for vascular disease among adults with previously diagnosed diabetes. JAMA, 2004. 291(3): p. 335-342.
21. Clark, W. The Benefits of Tight Control No End in Sight. Diabetes Self-Management Articles 2008 [cited 2014 Nov. 30]; Available from: http://www.diabetesselfmanagement.com/articles/blood-glucose-monitoring/the_benefits_of_tight_control/all/.
22. Khunti, K., et al., Clinical Inertia in People With Type 2 Diabetes A retrospective cohort study of more than 80,000 people. Diabetes care, 2013. 36(11): p. 3411-3417.
23. Global Markets for Diabetes Therapeutics and Diagnostics. 2013, BCC Research.
24. Crane, R., GI Dynamics Commences Trading on the ASX Following Successful A$80 Million Offers. 2011, GI Dynamics: www.investor.gidynamics.com. p. 1.
25. Shu, C. Fractyl Labs Raises $40M Series C To Develop Diabetes Treatments. 2014 Sep. 4 [cited 14 Nov. 30]; Available from: http://techcrunch.com/2014/09/04/fractyl-labs-raises-40m-series-c-to-develop-diabetes-treatments/.
26. GI Dynamics 2013 Annual Report. 2014, GI Dynamics: Online.
27. de Moura, E. G., et al. One Year Results of an Endoscopic, Duodenal-Jejunal Exclusion Device for Weight Loss and Control of Type 2 Diabetes. in OBESITY. 2011. Nature Publishing Group.
28. De Moura, E., et al., Six Months Results of the Duodenal-Jejunal Bypass Liner for the Treatment of Obesity and Type 2 Diabetes. J Gastroint Dig Syst S, 2012. 2: p. 2.
29. Koehestanie, P., et al., The Effect of the Endoscopic Duodenal-Jejunal Bypass Liner on Obesity and Type 2 Diabetes Mellitus, a Multicenter Randomized Controlled Trial. Annals of Surgery, 2014. 260(6): p. 984-992.
30. Dynamics, G., 2012 Annual Report. 2013.
31. Economic costs of diabetes in the US in 2012, in Diabetes Care. 2013, American Diabetes Association. p. 1033-1046.
32. Medicare's Coverage of Diabetes Supplies & Services, in www.medicare.gov, C.f.M.M. Services, Editor. 2013, US Department of Health and Human Services.
33. Association, A.D., Economic Costs of Diabetes in the U.S. in 2012, in Diabetes Care. 2013.
34. Turner, R., et al., Risk factors for coronary artery disease in non-insulin dependent diabetes mellitus: United Kingdom Prospective Diabetes Study (UKPDS: 23). BMJ, 1998. 316(7134): p. 823-828.
35. Gilmer, T. P., et al., The cost to health plans of poor glycemic control. Diabetes Care, 1997. 20(12): p. 1847-1853.
36. Pories, W. J. and R. J. Albrecht, Etiology of type II diabetes mellitus: role of the foregut. World Journal of Surgery, 2001. 25(4): p. 527-531.
37. Amiji, M., et al., Mucoadhesive vesicles for drug delivery. 2008, Google Patents.
38. Qian, Y., et al., Enteric-coated capsule containing cationic nanoparticles for oral insulin delivery. 2013, Google Patents.
39. Chang, L. C., et al., Innovative formulation for oral insulin delivery. 2009, Google Patents.
40. Harris, B. Z., et al., Mucoadhesive devices for delivery of active agents. 2013, Google Patents.
41. Berg, E. P. and D. B. Masters, Mucoadhesive drug delivery devices and methods of making and using thereof 2005, Google Patents.
42. Masters, D. B. and E. P. Berg, Mucoadhesive drug delivery devices and methods of making and using thereof 2013, Google Patents.
43. Economic costs of diabetes in the US in 2007, in Diabetes care. 2008, American Diabetes Association. p. 596-615.
44. Powell, A., Obesity? Diabetes? We've been set up, in Harvard Gazette. 2012, Harvard University: Online.
45. Rubino, F., et al., Diabetes Surgery: A New Approach to an Old Disease. Diabetes Care, 2009. 32(2).
46. Bellinger, D. A., E. P. Merricks, and T. C. Nichols, Swine models of type 2 diabetes mellitus: insulin resistance, glucose tolerance, and cardiovascular complications. ILAR journal, 2006. 47(3): p. 243-258.
47. Dodou, D., A. del Campo, and E. Arzt. Mucoadhesive micropatterns for enhanced grip. in Engineering in Medicine and Biology Society, 2007. EMBS 2007. 29th Annual International Conference of the IEEE. 2007. IEEE.
48. Tao, S. L. and T. A. Desai, Gastrointestinal patch systems for oral drug delivery. Drug Discovery Today, 2005. 10(13): p. 909-915.
49. Pinto, J. F., Site-specific drug delivery systems within the gastro-intestinal tract: from the mouth to the colon. International Journal of Pharmaceutics, 2010. 395(1): p. 44-52.
50. David, S., Delivery Systems for Biopharmaceuticals. J Pharm Pharmacol, 1992. 44(1): p. 5.
51. Selby, W., Complete small-bowel transit in patients undergoing capsule endoscopy: determining factors and improvement with metoclopramide. Gastrointestinal endoscopy, 2005. 61(1): p. 80-85.
52. Rubino, Francesco, et al. “The mechanism of diabetes control after gastrointestinal bypass surgery reveals a role of the proximal small intestine in the pathophysiology of type 2 diabetes.” Annals of surgery 244.5 (2006): 741.
53. Thazhath, Sony S., et al. “Glucose absorption in small intestinal diseases.” Expert review of gastroenterology & hepatology 8.3 (2014): 301-312.
54. Patel, Rajesh T., et al. “Surgical control of obesity and diabetes: The role of intestinal vs. gastric mechanisms in the regulation of body weight and glucose homeostasis.” Obesity 22.1 (2014): 159-169.
55. Nguyen, Nam Q., et al. “Upregulation of intestinal glucose transporters after Roux-en-Y gastric bypass to prevent carbohydrate malabsorption.” Obesity 22.10 (2014): 2164-2171.
56. Wang, Gary, et al. “Accelerated gastric emptying but no carbohydrate malabsorption 1 year after gastric bypass surgery (GBP).” Obesity surgery 22.8 (2012): 1263-1267.
57. Lips, Mirjam A., et al. “Roux-en-Y Gastric Bypass Surgery, but Not Calorie Restriction, Reduces Plasma Branched-Chain Amino Acids in Obese Women Independent of Weight Loss or the Presence of Type 2 Diabetes.” Diabetes care 37.12 (2014): 3150-3156.
58. Woo, Y. C., et al. “Fibroblast growth factor 21 as an emerging metabolic regulator: clinical perspectives.” Clinical endocrinology 78.4 (2013): 489-496.
59. Lips, Mirjam A., et al. “Calorie restriction and Roux-en-Y gastric bypass have opposing effects on circulating FGF21 in morbidly obese subjects.” Clinical endocrinology (2014).
60. PCT Patent Application Publication No. X02014151565 A1 to Karp, et al., for “Compounds to Modulate Intestinal Absorption of Nutrients.” published Sept. 25, 2014.

Although the foregoing subject matter has been described in some detail by way of illustration and example for purposes of clarity of understanding, it will be understood by those skilled in the art that certain changes and modifications can be practiced within the scope of the appended claims.

1. A method for applying a physical barrier to the gastrointestinal (GI) tract of a subject between the intestinal lining and the luminal contents, wherein the physical barrier has one or more of the following effects or characteristics: (a) is created in situ; (b) comprises one or more discrete and non-contiguous components; and (c) preserves significant nutrient absorption capacity within the intestines of the subject; and combinations thereof.

2. (canceled)

3. The method of claim 1, wherein the physical barrier regulates one or more neurohormonal signaling pathways.

4. The method of claim 1, wherein the physical barrier is created when a cationic compound combines in situ with the anionic mucins lining the wall of the intestines.

5. The method of claim 1, wherein the subject is afflicted by a metabolic disorder selected from the group consisting of obesity, pre-diabetes, insulin resistance, type 1 diabetes mellitus, type 2 diabetes mellitus, glucose impairment, hypertension, and dyslipidemia.

6. The method of claim 1, further comprising applying the physical barrier to limit an area of effective coverage on the lining of the GI tract to a section of the GI tract less than about 30 cm in length, wherein the section of the intestines begin at the pyloric sphincter and extends distally toward the jejunum.

7. The method of claim 6, wherein the physical barrier is applied to the GI tract starting in the duodenum with progressively increasing lengths from about 1 cm to about 30 cm to have a dose dependent effect.

8. The method of claim 1, wherein the physical barrier comprises a bioadhesive component.

9. The method of claim 8, wherein the bioadhesive component adheres to the mucosa of the GI tract for a retention period.

10-13. (canceled)

14. The method of claim 8, wherein the bioadhesive component comprises a layer having a thickness between about 0.1 microns and 1000 microns.

15. The method of claim 1, wherein the physical barrier further comprises a component selected from the group consisting of a semipermeable component, an impermeable component, an enteric component, and combinations thereof.

16. The method of claim 1, wherein the semipermeable or impermeable layer has a thickness between about 0.1 microns and 1000 microns

17. The method of claim 1, wherein the physical barrier comprises a plurality of discrete patches.

18. The method of claim 17, wherein the discrete patches comprise one or more discrete layers.

19. The method of claim 17, wherein the discrete patches have a diameter having a range between about 1 micron and 5,000 microns.

20. The method of claim 17, wherein the discrete patches are self-folding.

21. The method of claim 20, wherein the discrete self-folding patches include a swelling layer.

22. (canceled)

23. The method of claim 20, wherein the discrete self-folding patches include a non-swelling layer.

24. (canceled)

25. The method of claim 1, wherein the physical barrier comprises a plurality of discrete microspheres.

26-30. (canceled)

31. The method of claim 1, wherein the physical barrier or a formulation thereof further comprises a dissolvable substance, wherein the dissolvable substance dissolves at one or more specific sites of the gastrointestinal tract of the subject thereby applying the physical barrier thereto.

32-43. (canceled)

44. The method of claim 1, wherein the physical barrier, when compared to a sham control, does not result in a significant difference in blood d-xylose concentration as measured in a d-xylose concentration test.

45. (canceled)

46. The method of claim 1, wherein the physical barrier comprises about 1-5% by weight glycerine, about 0.1%-5% by weight polyacrylic acid polymer, about 0.1%-5% by weight potassium hydroxide, and about 1.5% by weight benzyl alcohol.

47. The method of claim 1, wherein the physical barrier comprises between about 0.1 wt/wt % to about 5.0 wt/wt % of one or more mucoadhesives, wherein the one or more mucoadhesive are selected from the group consisting polyvinylpyrrolidone, carboxymethylcellulose, dextran sulfate, hydroxyalkylcellulose, dermatan sulfate, a water-soluble vinyl polymer, chitosan, guar gum, xanthan gum, tragacanth gum, pectin, and polyacrylic acid.

48. (canceled)

49. The method of claim 1, wherein the physical barrier comprises about 0.35 wt/wt % of one or more mucoadhesives and about 5 wt/wt % of a viscosity inducing agent in an aqueous solution.

50. The method of claim 1, wherein the physical barrier comprises about 0.35 wt/wt % of a polyacrylic acid polymer, 1.5 wt/wt % of benzyl alcohol, 0.4 wt/wt % of sodium saccharin, and 0.05% wt/wt % of polysorbate 60 in an aqueous solution.

51. The method of claim 1, wherein the physical barrier comprises:
from about 0.001% to about 7% by weight of hyaluronic acid, or a pharmaceutically acceptable salt thereof, wherein the hyaluronic acid has a molecular weight between about 1.6 million Daltons and 2.2 million Daltons;
from about 0.04% to about 15% by weight of a K60 to K100 polyvinylpyrrolidone;
or a pharmaceutically acceptable salt thereof.

52. The method of claim 1, wherein the physical barrier forms a lining that when prepared at 10 mg/mL and applied to 1 cm2 surface area cellulose nitrate filter with 0.45 micron holes in a Franz diffusion chamber exhibits more than 60% permeation of glucose 5 minutes after addition of 120 g/L glucose solution to the top chamber at a pH of 1.

53. The method of claim 52, wherein the lining exhibits a permeation of glucose 5 minutes after addition of 120 g/L glucose solution to the top chamber at a pH of 1, and wherein the permeation has a range selected from the group consisting of between about 60% to about 100%, between about 65% to about 95%, between about 70% to about 90%, and from about 75% to about 85%.

54-55. (canceled)

56. A method for marketing a treatment for a metabolic disorder comprising packaging the treatment along with labeling that identifies the treatment as being useful to:
create a physical barrier in a subject between the intraluminal contents and the intestinal lining;
preserves significant nutrient absorption capacity in the proximal intestines of the subject; and/or
inhibit one or more neurohormonal pathways in the proximal intestines of the subject.
